(19) United States
(12) Patent Application Publication
(10) Pub. No.: US 2012/0244624 A1
(43) Pub. Date: Sep. 27, 2012

(54) ANALYZING METHOD AND SYSTEM FOR TEST STRIPS

(75) Inventor: Hsiung Hsiao, HsinChu (TW)

(73) Assignees: Middleland Sensing Technology Inc., HsinChu City (TW); Hsiung Hsiao, HsinChu City (TW)

(21) Appl. No.: 13/159,891

(22) Filed: Jun. 14, 2011

(30) Foreign Application Priority Data
Mar. 25, 2011 (TW) 100110431

(51) Int. Cl. G01N 33/84; G01N 33/50; G01N 33/68; G01N 33/52; G01N 33/64

(52) U.S. Cl. 436/86; 422/400; 436/128; 436/95; 436/163; 436/164

(57) ABSTRACT

An exemplary test strip reading and analyzing system includes a test strip unit, a mobile device and a remote computing apparatus. The test strip unit includes at least one tested-object reacting region. The mobile device includes an image capture module for capturing a test-strip image of the test strip unit and a transmission module electrically coupled to the image capture module. The remote computing apparatus, installed with a test strip analyzing system, is for analyzing the test-strip image and accordingly generating an test report. The test-strip image captured by the image capture module is transmitted to the remote computing apparatus through the transmission module; and the test report generated by the remote computing apparatus is consequently transmitted to the mobile device.

### FIELD OF THE INVENTION

The present invention relates to an analyzing system for test strips and an analyzing method thereof, and more particularly to an analyzing system for test strips and an analyzing method thereof realized by a mobile device.

### BACKGROUND OF THE INVENTION

On a conventional test strip there is a reacting region for presenting a corresponding color variation in response to a material concentration on a tested object while being attached to the tested object.

To use the conventional test strip, firstly a user needs to attach the reacting region on the tested object and wait the reaction between the reacting region and the tested object is done, and accordingly the user can obtain corresponding data via an analysis of the color variation presented on the reacting region of the test strip.

Because the process of color analyzing of the conventional test strip much relies on manual work, thereby some disadvantages such as time-consuming or a high possibility of artificially misreading are accordingly brought in. To overcome these disadvantages, some test strip reading and analyzing systems, capable of automatically generating test data through scanning the color variation on the reacting region and analyzing the scanned color, are developed today. However, for enhancing the analyzing accuracy of the test strip, the test strip reading and analyzing system with the automatic scanning and analyzing manners consequently also brings in some other disadvantages such as a larger machine size or a higher price, so as not easy to be published to a market. In addition, because the limits of machine size and price, only hospitals or professional test institutes have the ability to provide the related test service, thereby under a normal condition users have to go to hospitals or professional test institutes in person for the related tests, so not only cost users a lot of time for the test but also waste the society resource.

### SUMMARY OF THE INVENTION

Therefore, an objective of the present invention is to provide a test strip reading and analyzing system realized by a mobile device.

Another objective of the present invention is to provide a test strip reading and analyzing method corresponding to the aforementioned test strip reading and analyzing system.

A test strip reading and analyzing system comprises: a test strip unit comprised of at least one tested-object reacting region; a mobile device comprised of an image capture module which is for capturing a test-strip image of the test strip unit and a transmission module which is electrically coupled to the image capture module; and a remote computing apparatus installed with a test strip analyzing system which is for analyzing the test-strip image and accordingly generating an test report. The test-strip image captured by the image capture module is transmitted to the remote computing apparatus through the transmission module, and consequently the test report generated by the remote computing apparatus is transmitted to the mobile device.

In one embodiment, the above mentioned transmission of the test-strip image from the transmission module to the remote computing apparatus is based on an Internet, a cable transmission manner or a wireless transmission manner.

In one embodiment, the above mentioned test-strip image is automatically transmitted to the remote computing apparatus through the transmission module.

In one embodiment, the above mentioned test-strip image is manually transmitted to the remote computing apparatus from the transmission module through an operation to the mobile device.

In one embodiment, the above mentioned mobile device can be a mobile phone, a PDA, a digital camera, a pad computer or a notebook computer.

In one embodiment, the above mentioned mobile device further comprises pre-operation software. The pre-operation software is one of file transmission software, photograph software and user-interface software, or a combination of these software.

In one embodiment, the above mentioned test strip is a urine test strip or a water quality test strip.

A test strip reading and analyzing method in accordance with another embodiment of the present invention comprises steps of: providing a test strip unit which comprises at least one tested-object reacting region; providing a mobile device which comprises an image capture module and a transmission module; providing a remote computing apparatus which is installed with a test strip analyzing system; capturing a test-strip image of the test strip unit via the image capture module; transmitting the test-strip image to the remote computing apparatus through the transmission module; generating an test report after the test strip analyzing system performing an analysis to test-strip image; and transmitting the test report back to the mobile device.

In one embodiment, the above mentioned transmission of the test-strip image from the transmission module to the remote computing apparatus is based on an Internet, a cable transmission manner or a wireless transmission manner.

In one embodiment, the above mentioned transmission of the test-strip image from the transmission module to the remote computing apparatus is executed automatically.

In one embodiment, the above mentioned transmission of the test-strip image from the transmission module to the remote computing apparatus is executed manually.

To sum up, the present invention provides a test strip reading and analyzing system and a corresponding method realized by a mobile device. Via a communication between the mobile device and the remote computing apparatus installed with the test strip analyzing system, users can get the test report which is generated and transmitted from the remote computing system, thereby the advantages of the present invention such as saving users time, improving the test efficiency, reducing the cost of testing and analyzing the test strip, and reducing the waste of social resource are realized. In addition, based on the characteristic of the present invention, the test and analysis of the test strip can be widely applied to mobile electronic devices such as a mobile phone, a PDA, a digital camera, a pad computer, a notebook computer and a desktop computer, thereby it is easier to publish the test strip reading and analyzing system of the present invention to the market.

### BRIEF DESCRIPTION OF THE DRAWINGS

The above objects and advantages of the present invention will become more readily apparent to those ordinarily skilled in the art after reviewing the following detailed description and accompanying drawing, in which:

FIG. 1 is a schematic block diagram of a test strip reading and analyzing system in accordance with an embodiment of the present invention;

FIG. 2 is a schematic diagram of a test strip in accordance with an embodiment of the present invention; and

FIG. 3 is a flow chart of the test strip reading and analyzing method in accordance with an embodiment of the present invention.

### DETAILED DESCRIPTION OF PREFERRED EMBODIMENTS

The present invention will now be described more specifically with reference to the following embodiments. It is to be noted that the following descriptions of preferred embodiments of this invention are presented herein for purpose of illustration and description only. It is not intended to be exhaustive or to be limited to the precise form disclosed.

The disclosure relates to a test strip reading and analyzing system and a test strip reading and analyzing method.

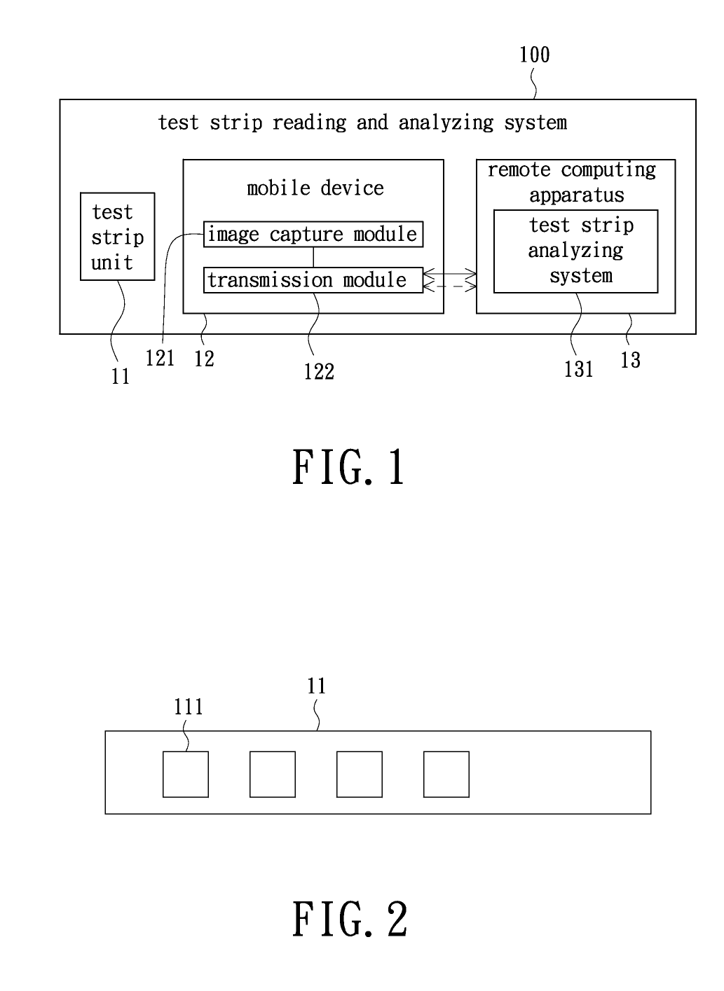

FIG. 1 is a schematic block diagram of a test strip reading and analyzing system in accordance with an embodiment of the present invention. FIG. 2 is a schematic diagram of a test strip in accordance with an embodiment of the present invention. As depicted in FIGS. 1 and 2, a test strip reading and analyzing system 100 comprises a test strip unit 11, a mobile device 12 and a remote computing apparatus 13. The test strip unit 11 comprises at least one tested-object reacting region 111. The mobile device 12 comprises an image capture module 121 which is configured to capture a test-strip image of the test strip unit 11 and a transmission module 122 which is electrically coupled to the image capture module 121. The remote computing apparatus 13, installed with a test strip analyzing system 131, is configured to analyze the test-strip image and accordingly generate a test report. In the embodiment, the remote computing apparatus 13 for example is a servo or a computer; the test-strip image captured by the image capture module 121 is transmitted to the remote computing apparatus 13 via the transmission module 122; and the test report generated by the remote computing apparatus 13 is transmitted back to the mobile device 12 via the transmission module 122.

In the embodiment, the test strip unit 11 is a color test strip; thereby the color presented on the tested-object reacting region 111 is varied with a reaction between the tested object (not shown) and the tested-object reacting region 111 during the test process. Basically, the color presented on the tested-object reacting region 111 is related to a material concentration or related features of the tested object. In the embodiment, the test strip unit 11 can be a urine test strip, and accordingly the multiple tested-object reacting regions 111 are used for testing items such as urine protein, urine ketene or urine glucose. In other embodiments, the test strip unit 11 can be a water quality test strip and accordingly the tested-object reacting regions 111 are used for testing items such as PH value, ion concentration or water hardness.

In the embodiment, the mobile device 12 is a portable device such as a mobile phone, a digital camera, a PDA (Personal Digital Assistant), a pad computer or a note computer. The image capture module 121 can be a photograph module or an image scanning module. The transmission module 122 can be a cable-manner or a wireless-manner transmission module, and is used for transmitting signals via the Internet, cable manner or a wireless manner.

In the test process of the present invention, a user firstly attaches the test strip unit 11 on the tested object, and consequently a color variation is presented on the test strip unit 11 in response to a reaction between the tested-object reacting region 111 and the tested object. Afterwards, the user uses the image capture module 121 of the mobile device 12 to capture the test-strip image of the test strip unit 11, and the test-strip image is then transmitted to the remote computing apparatus 13 via the Internet, the cable manner or the wireless manner. The test strip analyzing system 131 then analyzes the test-strip image and accordingly generates a corresponding test report. Via the transmission module 122, the test report is transmitted back to the mobile device 12 from the remote computing apparatus 13; thereby the user can read the test report via the mobile device 12.

In addition, the transmission of the test-strip image from the transmission module 122 to the remote computing apparatus 13 can be automatically executed in the embodiment after the test-strip image is captured by the image capture module 121. Or, in other embodiments, the transmission of the test-strip image from the image capture module 121 to the remote computing apparatus 13 can be manually executed after a user operating the mobile device 12.

In another embodiment, the mobile device 12 can be further installed with a pre-operation software (not shown); herein, the pre-operation software can be one of file transmission software, photograph software and user-interface software, or a combination of these software. Specifically, the user-interface software is used for routing the process of the test strip analyzing; in other words, after entering the user interface a user can efficiently complete the test strip analysis via the instructions provided by the user-interface software.

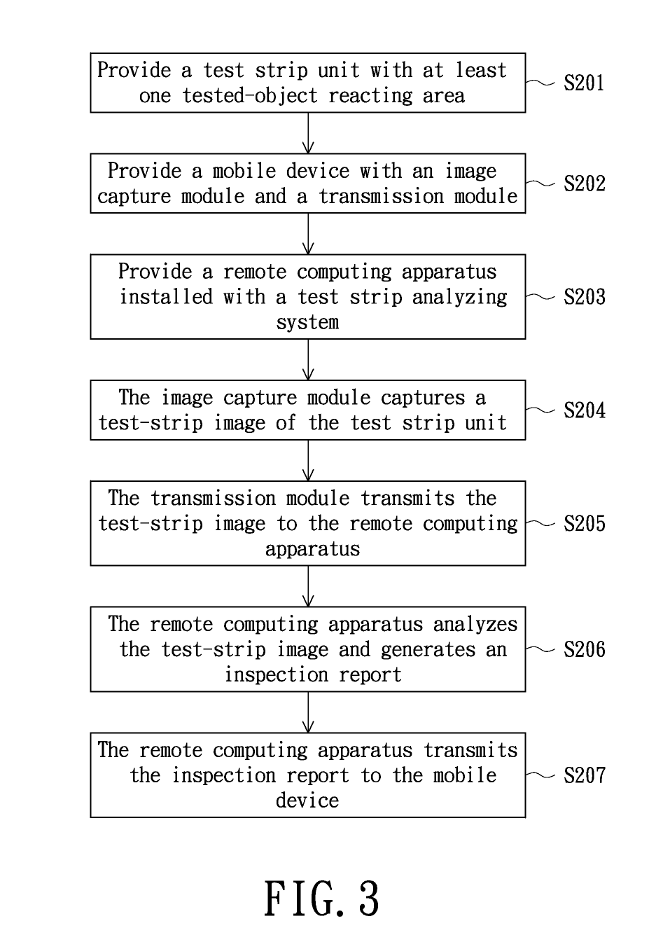

FIG. 3 is a flow chart of the test strip reading and analyzing method in accordance with an embodiment of the present invention. To get a clear understanding of the embodiment, please refer to the FIG. 3 together with the FIGS. 1 and 2. Following is the steps of the test strip reading and analyzing method in accordance with an embodiment of the present invention.

Step S201: provide a test strip unit 11 comprised of at least one tested-object reacting region 111. Herein, the test strip unit 11 is attached to the tested object and a color variation is presented on the tested-object reacting region 111 in response to a reaction between the tested-object reacting region 111 and the tested object. The test strip unit 11 is a urine test strip in the embodiment; or the test strip unit 11 can be a water quality test strip in other embodiment.

Step S202: provide a mobile device 12 installed with an image capture module 121 and a transmission module 122. Herein, the image capture module 121 is electrically coupled to the transmission module 122.

Step S203: provide a remote computing apparatus 13 installed with a test strip analyzing system 131. Step S204: captures a test-strip image of the test strip unit 11 via the image capture module 121. Step S205: transmit the test-strip image to the remote computing apparatus 13 via the transmission module 122. Step S206: analyze the test-strip image through the test strip analyzing system 131 and accordingly generate an test report. Step S207: transmit the test report back to the mobile device 12 from the remote computing apparatus 13.

In the step S205, the transmission of the test-strip image from the transmission module 122 to the remote computing apparatus 13 can be realized via the Internet, the cable or wireless transmission manner. In addition, the transmission of the test-strip image from the transmission module 122 to the remote computing apparatus 13 can be automatically executed after the test-strip image is captured by the image capture module 121. Or, the transmission of the test-strip image from the image capture module 121 to the remote computing apparatus 13 can be manually executed after an operation to the mobile device 12.

To sum up, the present invention provides a test strip reading and analyzing system and a corresponding method realized by a mobile device. Via a communication between the mobile device and a remote computing apparatus installed with a test strip analyzing system, users can get the test report which is generated and transmitted from the remote computing system, thereby the advantages of the present invention such as saving users time, improving the test efficiency, reducing the cost for testing and analyzing the test strip, and reducing the waste of social resource are realized.

In addition, based on the characteristic of the present invention, the test and analysis of the test strip can be widely applied to mobile electronic devices such as a mobile phone, a PDA, a digital camera, a pad computer, a notebook computer and a desktop computer, thereby it is easier to publish the test strip reading and analyzing system of the present invention to the market.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A test strip reading and analyzing system, comprising:
a test strip unit, comprising a tested-object reacting region;
a mobile device, comprising an image capture module which is for capturing a test-strip image of the test strip unit and a transmission module which is electrically coupled to the image capture module; and
a remote computing apparatus, installed with a test strip analyzing system which is for analyzing the test-strip image and accordingly generating an test report;
wherein the test-strip image captured by the image capture module is transmitted to the remote computing apparatus through the transmission module, and consequently the test report generated by the remote computing apparatus is transmitted to the mobile device.

2. The test strip reading and analyzing system according to claim 1, wherein a transmission of the test-strip image from the transmission module to the remote computing apparatus is based on an Internet, a cable transmission manner or a wireless transmission manner.

3. The test strip reading and analyzing system according to claim 1, wherein the test-strip image is automatically transmitted to the remote computing apparatus through the transmission module.

4. The test strip reading and analyzing system according to claim 1, wherein the test-strip image is manually transmitted to the remote computing apparatus from the transmission module through an operation to the mobile device.

5. The test strip reading and analyzing system according to claim 1, wherein the mobile device can be a mobile phone, a PDA, a digital camera, a pad computer or a notebook computer.

6. The test strip reading and analyzing system according to claim 1, wherein the mobile device further comprises a pre-operation software, the pre-operation software is one of a file transmission software, a photograph software and a user-interface software, or a combination of these software.

7. The test strip reading and analyzing system according to claim 1, wherein the test strip is a urine test strip or a water quality test strip.

8. A test strip reading and analyzing method, comprising steps of:
providing a test strip unit which comprises at least one tested-object reacting region;
providing a mobile device installed with an image capture module and a transmission module;
providing a remote computing apparatus installed with a test strip analyzing system;
capturing a test-strip image of the test strip unit via the image capture module;
transmitting the test-strip image to the remote computing apparatus through the transmission module;
generating an test report after the test strip analyzing system performing an analysis to test-strip image; and
transmitting the test report back to the mobile device.

9. The method according to claim 8, wherein a transmission of the test-strip image from the transmission module to the remote computing apparatus is based on an Internet, a cable transmission manner or a wireless transmission manner.

10. The method according to claim 8, wherein a transmission of the test-strip image from the transmission module to the remote computing apparatus is performed automatically.

11. The method according to claim 8, wherein a transmission of the test-strip image from the transmission module to the remote computing apparatus is performed manually.

12. The method according to claim 8, wherein the mobile device can be a mobile phone, a PDA, a digital camera, a pad computer or a notebook computer.

13. The method according to claim 8, wherein the mobile device further comprises a pre-operation software, the pre-operation software is one of a file transmission software, a photograph software and an user-interface software, or a combination of these software.

14. The method according to claim 8, wherein the test strip is a urine test strip or a water quality test strip.
